(19) United States
(12) Patent Application Publication
(10) Pub. No.: US 2007/0219272 A1
(43) Pub. Date: Sep. 20, 2007

(54) Novel method and compositions for treatment of tinnitus

(76) Inventors: Johan Stjernschantz, Mariehamn (FI); Helge Rask-Andersen, Uppsala (SE)

Correspondence Address: DINSMORE & SHOHL, LLP, 1900 CHEMED CENTER, 255 EAST FIFTH STREET, CINCINNATI, OH, 45202, US

(21) Appl. No.: 11/703,394

(22) Filed: Feb. 6, 2007

Related U.S. Application Data
(60) Provisional application No. 60/765,662, filed on Feb. 6, 2006.

(51) Int. Cl. A61K 31/19

(52) U.S. Cl. 514/570

(57) ABSTRACT

The invention is related to a novel method and compositions for the treatment or alleviation of tinnitus. The compositions which are to be administered locally into the middle ear cavity, or orally, contain a therapeutically effective and physiologically acceptable amount of lithium. The treatment may be continuous or intermittent.

The present invention relates to the field of pharmacological treatment or alleviation of disorders of the auditory organs, in particular tinnitus. A novel method as well as compositions for this purpose are presented.

### BACKGROUND OF THE INVENTION

Tinnitus

Tinnitus, the perception of sound in absence of acoustic stimulus, is a very common disorder amongst middle age and elderly people. As much as 10-30 percent of the middle age-elderly population suffers from some degree of tinnitus. Tinnitus can be peripheral (cochlear) or central. In the latter the sound generation is assumed to arise in the auditory pathways within the brain. Most patients suffer from tinnitus of cochlear origin which appears to be due to a disorder in the organ of Corti which contains the hair cells. These cells transform mechanical energy into electrochemical impulses for propagation to the brain. However, the pathophysiologic mechanism of tinnitus is poorly understood. Various causes of tinnitus may include acoustic trauma leading to permanent destruction e.g. of hair cells in the organ of Corti, microvasculopathies in the cochlea, toxic effects of drugs, and infections as well as presbyacusis. Often tinnitus is associated with hearing loss. There are other variants of tinnitus as well some of which are caused by disorders in the tympanic membrane and external ear and which often can be successfully treated, but usually tinnitus derived from the inner ear is incurable or difficult to treat. Tinnitus can be determined by audiological means, e.g. by matching the tinnitus sound at the right pitch. The perception of tinnitus is highly variable between individuals and assessment of tinnitus is also based on subjective methods.

Currently there are no clinically proven remedies for the treatment of tinnitus (Dobie, 1999), and a drug that could be used for alleviation of the tinnitus symptoms would thus be very desirable from a clinical point of view.

Anatomy and Physiology of the Ear

The ear is divided into three main parts; the external ear, the middle ear and the inner ear. The external ear consists of the auricle and the ear canal which ends at the tympanic membrane. The middle ear consists of the tympanic membrane, the tympanic cavity, the auditory ossicles and the Eustachian tube. The inner ear is also called the labyrinth because of its complex structure consists of sacs and tubules suspended in cavities of the petrous portion of the temporal bone. These structures contain a fluid called the endolymph, while the space between the membranous labyrinth and the bone is filled with the perilymph. The bony labyrinth consists of two parts; the vestibule which houses the saccule and the utricle with the semicircular canals, and the cochlea a spirally coiled structure. The sense of balance is located in the vestibule while the sense of hearing is located in the cochlea.

The cochlea is a two and three quarters coiled cavity in the bone containing a membranous structure filled with fluid. The cochlear membraneous structure comprises three cavities; the scala vestibuli connected to the oval window and the middle ear ossicles; the scala tympani connected to the round window facing the middle ear; and finally the scala media or the cochlear duct being part of the endolymphatic system. The scala vestibuli and scala tympani are parts of the perilymphatic system. The scala media filled with endolymph contains the sound perceiving organ, the organ of Corti, a complex structure containing hair cells which convert the hydromechanical energy into electrical signals, supporting cells, a basilar membrane and a tectorial membrane, as well as nerve fibers with origin in the spiral ganglion. From the spiral ganglion nerve fibers project to the brain for further processing of the auditory signals. The scala media also contains a highly vascularized structure called stria vascularis, and it is regarded that the endolymph of a large portion of the inner ear is formed in this structure.

The sound reaching the tympanic membrane of the middle ear will cause it to vibrate and the energy is then passed on to the oval window of the inner ear by means of the ossicles. The energy from the oval window causes a pressure wave in the scala vestibuli to be conveyed through the tip of the cochlea through an opening into the scala tympani which is connected to the round window at the middle ear. This pressure wave in the perilymphatic fluid system probably causes through the basilar membrane the hair cells to vibrate against the tectorial membrane thus transforming mechanical energy into electrochemical energy. Finally the energy from the external sound is released from the cochlea into the middle ear through the round window membrane.

The endolymphatic system of the cochlea is connected through ductus reuniens to the endolymphatic system of the sacculus in the vestibular organ. The sacculus is further connected to the utriculus joined by three semicircular canals. The sacculus, utriculus and the semicircular canals have a physiologic function in detecting movements and position and thus relate to the sense of balance. Disorders in this part of the inner ear usually cause symptoms of vertigo often associated with nausea. Both the utriculus and the sacculus are connected through a small canal called the endolymphatcic duct to the endolymphatic sac. The endolymphatic duct, a miniscule structure, has a very important function in that the endolymph is believed to be resorbed into the lymphatics and/or blood vessels in this structure.

Effects of a Prostaglandin F_{2α} Analogue on Tinnitus

It has previously been demonstrated that intratympanic injection of latanoprost, a prostaglandin F_{2α} analogue, may reduce tinnitus in patients suffering from Ménière's disease (Rask-Andersen et al., 2005). This is also the subject of a co-pending U.S. patent application Ser. No. 10/343,789.

In addition, latanoprost was shown to exert other beneficial effects on the inner ear in Ménière's disease, e.g. improved hearing and reduced vertigo. This led to further investigations regarding the effects of intratympanic injection of latanoprost by using several different concentrations of the drug injected into the middle ear. In a recent clinical study, latanoprost acid lithium salt was chosen, as this facilitates the formulation of latanoprost in aqueous solution (Latanoprost here means the isopropyl ester prodrug of latanoprost acid).

### SUMMARY OF THE INVENTION

It has now unexpectedly been found that intratympanic injection of the lithium salt of latanoprost acid (hereinafter named latanoprost lithium salt) exerts a favorable effect on tinnitus loudness in patients suffering from Ménière's disease, and it is reasonable to believe that this effect is present in patients suffering from tinnitus of other etiology as well. The present inventors consequently present a method for the treatment of tinnitus in a human subject, wherein a physiologically acceptable composition containing a therapeutically effective amount of lithium is administered to said subject, as defined in the attached claims, hereby incorporated by reference. The invention comprises both systemic and local administration, as well as compositions for such use.

### DESCRIPTION

Lithium, the third element in the periodic system, is an alkali metal with an atomic weight of 7.941. Lithium has been used extensively for the treatment of psychiatric diseases especially manic episodes. Usually lithium is used as an inorganic salt e.g. lithium carbonate or lithium sulphate and it is taken orally as pills. Lithium has a number of cellular effects, one being that it mimics effects of sodium (Na⁺). Thus the excitability of nerve cells may be affected. It has also effects on the phosphoinositol signaling pathway in nerve cells by interfering with the recycling of inositol. Furthermore, lithium may interact with the adenylate cyclase signaling pathways. Lithium has a very narrow therapeutic index in man and plasma concentrations should be within 0.5-1.5 mM. Lower concentrations are ineffective and with higher concentrations than 1.5 mM various side effects may arise. In their investigations, the present inventors have found that when the lithium salt of latanoprost was used at a concentration corresponding to about 1 mM, i.e. in the therapeutic range of lithium, a surprisingly strong tinnitus reducing effect was achieved which was stronger than expected with latanoprost only, and the present inventors believe that the marked effect, if not totally, at least partly may be due to an effect of lithium when the latanoprost lithium salt dissociates in the ear.

Accordingly, the present invention makes available a method for the treatment of tinnitus in a human subject, wherein a physiologically acceptable composition containing a therapeutically effective amount of lithium is administered to said subject. The lithium is preferably used in the form of a salt of a prostaglandin, in particular a PGF_{2α} analogue. According to one embodiment, said prostaglandin is latanoprost acid.

According to another embodiment, the lithium is used in the form of an inorganic salt thereof.

According to another embodiment, said physiologically acceptable composition containing a therapeutically effective amount of lithium is administered orally, in the form of tablets, capsules or solutions.

In particular, the present invention makes available a method for the treatment of tinnitus in a human subject, wherein a physiologically acceptable composition containing a therapeutically effective amount of lithium is administered to the round and/or oval window of the inner ear of said subject. According to one embodiment, the physiologically acceptable composition containing a therapeutically effective amount of lithium is administered to the inner ear 1-100 times a year or continuously by using a pump device.

According to another embodiment, the lithium salt is administered to the inner ear using a soluble or solid slow release drug insert.

As lithium is a known therapeutic agent, albeit not suggested for the treatment or alleviation of tinnitus, the present inventors present the second medical use thereof. Consequently, the present invention also concerns the use of lithium, preferably a lithium salt, for the manufacture of a pharmaceutical composition for the treatment of tinnitus. Preferably said lithium salt is a salt of a prostaglandin, in particular an analogue of PGF_{2α}. According to one embodiment, the prostaglandin is latanoprost acid.

According to another embodiment, the lithium is used in the form of an inorganic salt thereof.

The invention also makes available a composition for the treatment of tinnitus, wherein said composition comprises a therapeutically active and physiologically acceptable amount of lithium in a mixture with a vehicle including a slow release matrix suitable for administration into the middle ear cavity. Said lithium is preferably present in the form of a lithium salt, an inorganic salt or a salt of a prostaglandin, in particular an analogue of PGF_{2α}. Said prostaglandin is preferably latanoprost acid.

### EXEMPLIFICATION OF THE INVENTION

The invention is exemplified by the following non-limiting example based on a recently performed clinical trial: Patients suffering from unilateral Ménière's disease were enrolled in a randomized, double blind, placebo-controlled clinical trial. The study followed a cross-over protocol. The patients received an intratympanic injection (injection into the middle ear cavity) of latanoprost lithium salt or placebo once daily during the three first days of each treatment period. The effect on hearing and tinnitus loudness was measured on Days 5, 15 and 29. Thereafter there was a treatment-free cross-over period of 2 months. After the cross-over period the patient entered the second treatment period and latanoprost lithium salt or placebo was again administered by intratympanic injection once daily during the three first days, and the same measurements of hearing and tinnitus were repeated. Latanoprost lithium salt was tested at 50 μg/ml, 150 μg/ml and 450 μg/ml concentration, and in addition one group of patients received latanoprost (isopropyl ester) at 50 μg/ml concentration according to the same cross-over study protocol.

The effect of latanoprost lithium salt and latanoprost (isopropyl ester) on tinnitus loudness is presented in Table 1. As can be seen from the results, both latanoprost lithium salt (450 μg/ml) and latanoprost (isopropyl ester; 50 μg/ml) reduced tinnitus loudness, but the reduction was significantly stronger with the lithium salt and furthermore statistically significant at all time points with the lithium salt. Although the concentration of latanoprost lithium salt was 9 times higher than the concentration of latanoprost (isopropyl ester) we believe that the main additional effect (compared with latanoprost) is based on the lithium salt because the lipophilicity of latanoprost (isopropyl ester) is more than 1000 times higher than the lipophilicity of the latanoprost lithium salt. Consequently the ester can be expected to traverse biological membranes such as the round window membrane much better than the water-soluble salt. Hence it is very likely that the difference in effect between the lithium salt and the isopropyl ester of latanoprost is accounted for by the lithium. The concentration of 450 μg/ml of latanoprost lithium salt corresponds to approximately 1 mM lithium, i.e. a concentration within the therapeutic range of lithium. The lower concentrations 150 μg/ml and 50 μg/ml of latanoprost lithium salt had no effect which can be anticipated if the effect on tinnitus loudness at least partly is based on the pharmacological effect of lithium since the two lower concentrations are below the therapeutic concentration range of lithium.

TABLE 1
Reduction in tinnitus loudness after intratympanic injection of
latanoprost lithium salt and latanoprost (isopropyl ester)
in patients with Ménière's disease

| | | Reduction of tinnitus loudness in decibels | | |
|---|---|---|---|---|
| Latanoprost | n | Day 5 | Day 15 | Day 29 |
| Latanoprost acid | 8 | −10.8 ± 3.5 | −15.4 ± 6.4 | −10.5 ± 3.9 |
| lithium salt | | (p = 0.0217) | (p = 0.0544) | (p = 0.0365) |
| Latanoprost | 5 | −5.5 ± 2.7 | −9.5 ± 5.8 | −9.6 ± 4.6 |
| isopropyl ester | | (NS) | (NS) | (NS) |

NS = not significant
Note:
The calculation of the reduction of tinnitus loudness is based on comparison to placebo taking into account differences in baseline before the active treatment and placebo treatment. The days indicate number of days after initiation of the treatment. (Mean ± SEM). Statistical significances are based on ANOVA.

Since lithium is known to exert a metabotropic effect on neural elements it appears as a clear possibility that lithium by itself may exert an effect on tinnitus loudness that could be clinically useful. Although we firmly believe that lithium exerts a beneficial effect on tinnitus, it appears based on this study and a previous clinical trial (Rask-Andersen et al., 2005) that the latanoprost isopropyl ester (in the absence of lithium) also exerts a beneficial effect on tinnitus. Thus latanoprost and lithium together may have a synergistic or at least additive effect on tinnitus.

Based on the above results, lithium is considered to exert a pharmacologic effect in the inner ear that is beneficial for the reduction of tinnitus. Lithium can be administered by direct injection/infusion as a salt of a prostaglandin or another pharmacologically active compound into the middle ear, or even as ear drops into the ear canal particularly when tubes have been installed into the ear drums. Lithium can also be used as an inorganic salt. In addition lithium can be administered orally as tablets/capsules or solution. If lithium is administered orally it should be on a daily basis, or several times a week. If lithium is injected into the middle ear less frequent administration is sufficient, e.g. from a few times a month to a few times a year depending on the pharmaceutical formulation and the severity of the tinnitus symptoms.

Pharmaceutical Compositions and Dosages

The preferred mode of administration of the pharmacologically active lithium compound is by direct injection through the tympanic membrane or by slow infusion using a pump into the middle ear. Pharmacologically active lithium compound encompasses prostaglandin lithium salt, other lithium salts of medicinal molecules with therapeutic effect as well as inorganic salts of lithium, e.g. lithium carbonate and lithium sulphate. Pharmaceutical compositions comprise the pharmacologically active principle dissolved in compatible vehicles for use in the middle ear. Such vehicles may comprise aqueous solutions, certain oil solutions and physiologically suitable and compatible preparations depending on the organic lithium salt, as well as gels, e.g. gels based on hyaluronic acid, or chondroitin sulphate and other glucosamine glycans. In particular crosslinked hyaluronic acid gels to prevent decomposition of the gel may be useful in order to establish a slow release formulation so that the lithium is released into the inner ear during a sustained period of time e.g. during several weeks, or months, or even longer periods. The vehicle furthermore may contain solubilizers, liposomes and physiologically compatible polymeres, e.g. polyvinylalcohole, hydroxymethyl-cellulose, hyaluronic acid, chondroitin sulphate and other glucosaminoglycans, to increase viscosity. Furthermore physiologically compatible nanoparticle formulations may also be employed. The formulations may be preserved with compatible preservatives in suitable concentrations for use in the middle ear. If lithium is given orally the daily dose should be adjusted to result in plasma concentrations of lithium within 0.5-1.5 mM.

The lithium containing medicament when administered by intratympanic injection should be given from 1-5 times a month to a few times a year. A slow release system either soluble or solid placed in the middle ear may be optimal to avoid repeated puncture of the ear drum. Alternatively installment of a tubing to the round window niche and a pump may be employed for administration of the new medicament. It may even be possible to administer the new medicament as ear drops through the ear canal if tubes in the ear drums have been installed or a middle ear wick between the eardrum and the round window is being employed. Local administration of the new medicament is preferable but if tinnitus is bilateral or of central origin lithium containing tablets, capsules or solutions may be used provided the plasma concentration of lithium is regularly monitored to avoid adverse effects of too high concentrations.

### REFERENCES

Dobie, R. A. (1999). A review of randomized clinical trials in tinnitus. Laryngoscope. 109: 1202-1211.

Rask-Andersen, H., Friberg, U., Johansson, M. and Stjernschantz, J. (2005). Effects of Intratynpanic injection of latanoprost in Meniere's disease: A randomized, placebo-controlled, double-blind, pilot study. Otolaryngology-Head and Neck Surgery. 133: 441-443.

1. A method for the treatment of tinnitus in a human subject, characterized in that a physiologically acceptable composition containing a therapeutically effective amount of lithium is administered to said subject.

2. The method according to claim 1, wherein the lithium is used in the form of a salt of a prostaglandin, in particular a PGF_{2α} analogue.

3. The method according to claim 2, wherein said prostaglandin is latanoprost acid.

4. The method according to claim 1, wherein the lithium is used in the form of an inorganic salt thereof.

5. The method for the treatment of tinnitus according to claim 1, wherein said physiologically acceptable composition containing a therapeutically effective amount of lithium is administered orally, in the form of tablets, capsules or solutions.

6. A method for the treatment of tinnitus in a human subject, characterized in that a physiologically acceptable composition containing a therapeutically effective amount of lithium is administered to the round and/or oval window of the inner ear of said subject.

7. The method according to claim 6, wherein the physiologically acceptable composition containing a therapeutically effective amount of lithium is administered to the inner ear 1-100 times a year or continuously by using a pump device.

8. The method according to claim 6, wherein the lithium salt is administered to the inner ear using a soluble or solid slow release drug insert.

9. The use of a lithium salt for the manufacture of a pharmaceutical composition for the treatment of tinnitus.

10. The use according to claim 9, wherein the lithium is a salt of a prostaglandin, in particular an analogue of PGF_{2α}.

11. The use according to claim 9, wherein the prostaglandin is latanoprost acid.

12. The use according to claim 9, wherein the lithium is used as an inorganic salt.

13. The use according to claim 9, wherein the pharmaceutical composition is in the form of tablets, capsules or solutions for the treatment of tinnitus.

14. A composition for the treatment of tinnitus, wherein said composition comprises a therapeutically active and physiologically acceptable amount of lithium in a mixture with a vehicle including a slow release matrix suitable for administration into the middle ear cavity.

15. A composition according to claim 14, wherein said lithium is present in the form of a lithium salt.

16. The use according to claim 14, wherein the lithium is a salt of a prostaglandin, in particular an analogue of PGF_{2α}.

17. The use according to claim 16, wherein the prostaglandin is latanoprost acid.

18. The use according to claim 14, wherein the lithium is used as an inorganic salt.
